(19) United States
(12) Patent Application Publication
(10) Pub. No.: US 2005/0244522 A1
(43) Pub. Date: Nov. 3, 2005

(54) Permeation enhancer comprising genus Curcuma or germacrone for transdermal and topical administration of active agents

(76) Inventors: Dario Norberto R. Carrara, Oberwil (CH); Arnaud Grenier, Steinbrunn-Le-Haut (FR); Celine Besse, Huningue (FR); Ingo Alberti, Huningue (FR); Laetitia Henry, Saint Louis (FR)

Correspondence Address: WINSTON & STRAWN LLP, 1700 K STREET, N.W., WASHINGTON, DC, 20006, US

(21) Appl. No.: 11/114,537

(22) Filed: Apr. 26, 2005

Related U.S. Application Data
(60) Provisional application No. 60/567,037, filed on Apr. 30, 2004.

(51) Int. Cl. A61K 35/78

(52) U.S. Cl. 424/756

(57) ABSTRACT

A formulation, method and system for the topical, transdermal or transmucosal administration of a therapeutically effective active agent. Particularly, the invention provides a formulation, system and method for enhancing the permeation or penetration of active agents across the dermal or mucosal surfaces of a mammalian subject. The formulation includes a plant extract of the genus Curcuma of the family Zingiberaceae, a germacrone, or a natural or synthetic constituent thereof, which has been found to increase penetration of the active agent across the dermal or mucosal surface. If desired, a secondary permeation enhancer of a polyalcohol, a monoalkyl ether of diethylene glycol, a tetraglycol, or a mixture thereof can be used for certain active agents for optimal permeation enhancement.

### CROSS REFERENCE

This application claims the benefit of U.S. Provisional Patent Application Ser. No. 60/567,037, filed Apr. 30, 2004, the contents of which are incorporated herein by reference thereto.

### FIELD OF THE INVENTION

The present invention relates to formulations, methods and systems for transdermal and transmembrane delivery of pharmaceutically active agents, such as drugs to mammalian subjects. In particular, the invention relates to permeation enhancers comprising plant extract of genus Curcuma or germacrone for enhancing penetration of active agents across mammalian dermal and mucosal surfaces.

### DESCRIPTION OF RELATED ART

Transdermal and/or transmucosal delivery of active agents provide a convenient, pain-free, and non-invasive method of administering active agents to a subject. Additionally, the administration of active agents, such as drugs, through the skin or mucosal surface avoids the well-documented problems associated with the “first pass effect” encountered by oral administration of active agents. As known in the art, orally administered drugs are absorbed and enter the bloodstream where they are transported by the portal vein directly to the liver before entering the general circulation of the body. The absorbed drug is then exposed to the liver during its first pass through the body. If the drug is subject to a high hepatic clearance, i.e., it is rapidly metabolized by the liver, then a substantial fraction of the absorbed dose is extracted from the blood and metabolized before it ever reaches the systemic circulation. The consequence of this “first pass effect” phenomenon is a significant reduction in the bioavailability of the drug. In some instances, the first pass effect is so large as to render oral administration of a drug ineffective.

Although the transdermal and/or transmucosal delivery of active agents overcome some of the problems associated with oral administration of active agents, such as that described above, they are not free of their own drawbacks. A major drawback of transdermal delivery systems is the limitation of the amount of active agent that can be transported across the skin or mucosal surface. This limitation is due to several factors. First, since the skin is a protective barrier by nature, the rates of transport of most compounds through the skin are quite slow. It is generally accepted that a patch surface beyond 50-100 cm² would result in difficulty of application. Therefore, the application of a transdermal semisolid dosage form such as a gel, cream, ointment, patch, liquid, and the like, augments the patient's compliance and the surface of application can be extended.

In order to overcome the barrier properties of the skin or mucosal surfaces and facilitate the percutaneous absorption of the active agent, it is known to use penetration enhancers. Substances such as azone, glycol, pyrrolidone, fatty alcohol, fatty acid and fatty esters are known for enhancing the penetration of active agents. See, “Pharmaceutical Skin Penetration Enhancement”, Marcel Dekker, New York 1993, pages 229-242, the content of which is incorporated herein by reference. Similarly, EP 0 367 431, the content of which is incorporated herein by reference, discloses that aliphatic alcohols such as isopropyl alcohol and isobutyl alcohol enhance the rate of transdermal delivery of steroid drugs.

In contrast, essential oils have been used for centuries to provide fragrance and flavorings to a wide variety of preparations, formulations and products. For example, essential oils have been used widely in perfumes, cosmetics, and foods.

Generally, essential oils are very complex substances, which contain numerous compounds. Examples of extractions of essential oils are disclosed in Perfumer and Flavorist, 17, November/December 1992, the content of which is incorporated herein by reference. Recently, some of the essential oils have been chemically synthesized.

Most uses of essential oils are as flavorings for foods and candies and as aromatics for bath, cosmetic and perfume products. For example, Zarif discloses the use of cinnamon oil as flavor substance in U.S. Pat. No. 6,592,894 and Gozu Yoko and Sakai Keiko disclose in WO 02/051428, the contents of each of which are incorporated herein by reference, a perfume composition containing fennel oil, grapefruit oil, pepper oil, hyssop oil, sege oil, estragon oil, eucalyptus oil, rosemary oil, cinnamon oil, clove oil, ylang ylang oil, ginger oil, geranium oil, or olibanum, and/or contains one or more members selected among limonen, pinen, myrcene, and benzyl benzoate, which are active ingredients contained in those oils.

Essential oils have also been used as delivery vehicles and active agents. Southard and Yemey disclose a method and composition for the treatment of Female Sexual Arousal Disorder in U.S. Patent Application No. 2003/0130183, the content of which is incorporated herein by reference, in which cinnamon oil, ginger oil, peppermint oil and other oil is utilized as a delivery vehicle. Meanwhile, numerous publications have disclosed the use of essential oils as active agents. For example, rosemary oil, anise oil, cinnamon oil, clove oil, lemon oil and cardamom oil have been shown to have antibacterial activity. See Elkhoully et al, 1980, Aust. J. Pharm. Sci., 9(3) September. 1980 and Pharm. Acta Helv., 66(9-10) 1991), the contents of each of which are incorporated herein by reference. However, this activity was minimal when compared with a preservative used in current pharmaceuticals. U.S. Pat. Nos. 4,927,816, 6,589,543 and 6,607,756, the contents of each of which are incorporated herein by reference, disclose that cinnamon oil, clove oil or ginger oil serve to stimulate the saliva production or to activate natural defense or to treat edema.

Additionally, menthol, eucalyptus oil, camphor, peppermint oil and wintergreen oil are currently used in over-the-counter topical preparations such as BenGay, Mineral Ice, Flexall 454, etc. at concentrations as high as 30%. These topical medications assert pain relief but, according to the FDA, they act to relieve pain by producing a counter-irritation, and not by penetrating the skin and acting systemically to reduce inflammation and swelling which are the causes of pain. Additionally, medications such as those described above do not profess to reduce bruising, deep pain, itching, or induce wound healing.

It has been known that some essential oils are capable of penetrating skin. For instance, Williams and Barry disclose that chenopodium, eucalyptus, anise and ylang ylang oils penetrate the skin. 1989, Internat. J. Pharm., 57, 1989, the content of which is incorporated herein by reference. Thus, some natural oils have been tested for permeation enhancing behavior. For example, in 1990, Carelli et al. examined mink oil, turtle oil, and their ethylated forms as permeation enhancers. Using caffeine, salicylamide and 2-hydroxy-4-methoxybenzophenone as model drugs and hairless mouse skin as a model membrane, it was found that the more polar drugs, such as caffeine, followed by salicylamide, were most sensitive to the enhancing effect of these oils. The oils permeation-enhancing activity appears to derive primarily from an enhancement of the diffusion coefficient of the drug across the skin layer, rather than an increase in drug solubility.

Further, eucalyptus, peppermint and turpentine oils are disclosed by Sinha as permeation enhancers. See, “Permeation Enhancers for Transdermal Drug Delivery,” Drug Dev Ind Pharm, 2000 November; 26(11): pages 1131-1140, the content of which is incorporated herein by reference. Eucalyptus oil was found to be the most effective on 5-fluorouracil rat skin permeation.

Morimoto describes in U.S. Pat. No. 5,240,932, percutaneous absorbable compositions of morphine or analgesics of morphine containing an essential oil such as mentha oil and peppermint oil used as percutaneous absorption accelerator. Similarly, Cody et al disclose in U.S. Pat. No. 5,807,568, a topical composition containing flurbiprofen and the use of peppermint oil and eucalyptol as enhanced delivery agents. Further, U.S. Pat. No. 5,229,130 to Sharma discloses vegetable oil-based permeation enhancer compositions. A permeation enhancing amount of a mixture of vegetable oils selected from the group consisting of almond oil, babassu oil, castor oil, Clark A oil, coconut oil, corn oil, cotton seed oil, jojoba oil, linseed oil, mustard oil, olive oil, palm oil, peanut oil, safflower oil, sesame oil, soybean oil, sunflower-seed oil and wheat germ oil, wherein the drug is present in the composition in any amount in the range of from about 1% to about 10% by weight and the mixture of vegetable oils is present in an amount of about 5% to about 25% by weight. The mixture of vegetable oils preferably was a mixture of coconut oil and soybean oil.

Accordingly, although essential oils are mostly used as fragrance, flavorings, and active agents, some essential oils have been reported as having penetration enhancing behavior.

Curcuma zedoaria is a native plant of Ceylon, Indonesia and the East India islands. Presently, Curcuma is widely cultivated in India, South East Asia, China, and Brazil. Curcuma Zedoaria oil is extracted from the rhizome, leaf, root, bulb, stem, flower, bark, inflorescence or seed of Curcuma Zedoaria Roscoe plant. Curcuma Zedoaria is a species of the genus Curcuma of the family Zingiberaceae. The genus Curcuma comprises many other species, including zedoaria, as well as sub-species.

The Zedoary root is a popular spice and medicine in the Eastern world, used similarly to its cousin turmeric (Curcuma longa L.) in condiments and curries. Traditionally, zedoary is used for treating flatulent colic and indigestion. However, in addition to the well-known effect of zedoary as a stomachic, dehydrocurdione, the major sesquiterpene found in Curcuma zedoaria roots has anti-inflammatory potency related to its antioxidant effect.

In addition to gastrointestinal remedies, essential oils obtained from plant extracts of the Curcuma and in general of the Zingiberaceae family, such as curcumin and other curcuminoids, are known to be useful for the treatment and prophylaxis of other disorders or diseases, including antiviral agents (EP568001); anti-inflammatory agents (EP440885); hair loss (EP319058) malabsorption syndromes (EP256353); platelet anti-aggregation, and anti-cholesterol agents (U.S. Pat. Nos. 5,108,750; 4,906,471, 4,842,859); hepatitis B and AIDS (WO88/05304); neurological disorders (WO88/08713), hair loss and vasokinetic, antibacterial and antiseborrhoic activities (U.S. Pat. No. 6,270,752); hair stimulant (WO 96/10387); excessive formation of leucotrienes and/or prostaglandins (U.S. Pat. No. 5,401,777); musculoskeletal disease (U.S. Pat. No. 5,494,668) and antivenin agents (U.S. Pat. No. 4,568,546). Each patent is incorporated herein by reference thereto.

Additionally, Zedoary oil is also used in Chinese medicine as an anti-cancer agent (WO 02078722); for injection in nano capsule freeze dried ampoule powder (CN 1410079) and in capsule for resisting virus (CN 1400006). The production of curcumin, curcuma oil and zedoary alcohol is described in CN 1036026, the content of which is incorporated herein in its entirety by reference thereto. Further, the contents of each patent discussed herein is incorporated herein in its entirety by reference thereto.

Moreover, Curcuma Zedoaria oil is also used in various cosmetic formulations as an excipient or fragrance in massage oils or in shampoos and as lymphatic stimulator in gommage cream. Further, the anti-oxidizing and dyeing properties of curcumin and of the derivatives promote its wide use in the food and cosmetic industries as natural preservatives (U.S. Pat. No. 5,266,344).

While there are a number of patents and publications which relate to the use of a variety of permeation enhancers, there is a need for an improved permeation enhancer. The present invention now satisfies this need.

### SUMMARY OF THE INVENTION

The purpose and advantages of the present invention will be set forth in and be apparent from the description that follows, as well as will be learned by practice of the invention. Additional advantages of the invention will be realized and attained by the practice of the formulations and methods particularly pointed out in the written description and claims hereof, as well as from the appended drawings.

To achieve these and other advantages and in accordance with the purpose of the invention, as embodied and broadly described, the invention provides a formulation for the topical, transdermal or transmucosal administration of a therapeutically effective active agent. Particularly, the invention provides a formulation and method for enhancing the permeation or penetration of active agents across the dermal or mucosal surfaces of a mammalian subject. It has been surprisingly found that a formulation comprising an active agent and a plant extract of the genus Curcuma or a natural or synthetic constituent thereof increases penetration of the active agent across the dermal or mucosal surfaces. It has also been found that the formulation of the invention also increases the absorption rate of the active agent and allows the steady-state drug flux to be reached in a relatively short time. Advantageously, the formulation of the invention induces a more rapid onset of action of the active agent.

Additionally, it has been surprisingly found that a formulation including germacrone or a derivative thereof also increases the penetration of active agent across the dermal or mucosal surfaces. The germacrone can be extacted from many plants including Curcuma as discussed below.

In one aspect of the invention is provided a pharmaceutical formulation comprising a therapeutically effective amount of an active agent, and a permeation enhancer comprising a plant extract of the genus Curcuma or a natural or synthetic constituent thereof in an amount sufficient to enhance the permeation of the active agent through mammalian dermal or mucosal surfaces.

The plant genus Curcuma is of the family Zingiberaceae, and comprises numerous species and constituents, which can be extracted from Curcuma plant or more specifically from the oil. For example, the species of the genus Curcuma can be selected from the group including Curcuma zedoaria, Curcuma alismatifolia, Curcuma amada, Curcuma angustifolia, Curcuma aromatica, Curcuma cordata, Curcuma petiolata, Curcuma elata, Curcuma flaviflora, Curcuma gracillima, Curcuma harmandii, Curcuma longa, Curcuma ornata, Curcuma roscoeana, Curcuma sparganifola, and Curcuma thorelii or a combination thereof. Preferably, the permeation enhancer comprises a natural or synthetic extract of Curcuma zedoaria. The constituents extracted from Curcuma zedoaria can be selected from the group consisting of zedoarofuran, 4-epicurcumenol, neocurcumenol, gajutsulactones A and B, zedoarolides A and B, germacrone-4,5-epoxide, germacrone, furanodienone, curzerenone, zedereon, dehydrocurdione, curcumenol, isocurcumenol, curcumenone, curmanolide A, and curmanolide B, or a combination thereof. Additionally, the permeation enhancer may comprise a mixture of plant extracts of the constituents, species, sub-species and/or genus Curcuma.

The extract may be natural or synthetic. In this regard, the Curcuma Zedoaria can be extracted from the rhizome of Curcuma Zedoaria Roscoe plant. Alternatively, the extract can be taken from any other part of the plant, i.e., leaf, root, bulb, stem, flower, bark, inflorescence and seed. Preferably, the extract is present in the form of an oil in an amount of about 0.10% to 15% of the formulation by weight.

Extraction methods for extracting volatile oils, especially the oil of genus Curcuma is well described in literature. For example, U.S. Pat. No. 6,344,575 to Rubin discloses the process of extraction of volatile oils from plant material, such as tumeric (Curcuma spp.). According to the Rubin method, freshly collected plant material is subjected to mechanical working. The worked plant material is boiled. Thereafter, the distillate is collected and separated into its components. Additionally, U.S. Pat. No. 5,176,913 to Honerlagen describes a process for preparing a partial extract containing the volatile in steam components and further lipophilic components of medical plants and/or species plants or parts thereof, which may be charted in fresh or dried state. Numerous plants are extracted, especially Curcuma zeodaria, according to this process, as are further sub species (Rhizoma). Each of the Rubin and Honerlagen patents are expressly incorporated herein by reference thereto. The Curcuma zedoaria can be obtained from western Nepal or any other geographic origin or varieties. Curcuma zedoaria is a native plant of Ceylon, Indonesia and the East India islands, but is presently widely cultivated in India, South East Asia, China and Brazil.

Alternatively, the plant extract can be synthetically prepared by methods known in the art. For the purpose of illustration, the isolation of the constituents from the zedoaria rhizoma variety is described by H. Matsuda in Chem. Pharm. bull. 49(12) 1558-1566 (2001), the entire content of which is incorporated herein by reference thereto.

The formulation may further include a delivery vehicle. The delivery vehicle of the invention may comprise at least one of a C2 to C4 alkanol, a polyalcohol and water. For purpose of illustration the C2 to C4 alkanol may include ethanol, isopropanol, n-propanol, butanol, and preferably ethanol; and the polyalcohol may include propylene glycol, butylene glycol, hexylene glycol, ethylene glycol, polyethylene glycol, and preferably propylene glycol. Preferably, the water is purified water.

Additionally, the formulation may include a tetraglycol furol or a monoalkyl ether of diethylene glycol. For example, the preferred tetraglycol furol is glycofurol having the formula: (C₂H₄O)mult-C₅—H₁₀—O₂, and is represented by the formula:

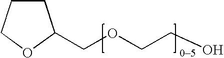

The preferred monoalkyl ether of diethylene glycol is diethylene glycol monoethyl ether or diethylene glycol monomethyl. Most preferably, diethylene glycol monoethyl ether is used.

The formulation of the present invention may further include at least one of the following: a gelling agent or viscosant, a pH regulator or neutralizing agents, preservatives, antioxidants, buffers, humectants, sequestering agents, moisturizers, surfactants, emollients, solubilizers, solvents, emulsifiers, skin protectants, essential oils, fragrances, flavors, and any combinations thereof. The amounts of such additives can be readily determined by one skilled in the art, according to the particular properties desired.

The formulation may be in a form for topical, transdermal or transmucosal administration. Preferably, the formulation is a spreadable, semi-solid, jelly-like gel. Alternatively, however, the formulation may be in the form of a spray, ointment, aerosol, patch, foam, buccal and sublingual tablets, suppositories, vaginal dosage form, or other passive or active transdermal devices for absorption through the skin or mucosal surface.

The formulation of the present invention may be applied directly to the skin such as by, for example and not limitation, a gel, lotion, solution, emulsion, ointment, foam, microsphere, nanosphere, microcapsule, nanocapsule, nail lacquer or cream or indirectly through a patch, bandage, or other occlusive dressing.

As any active agent may be used, the formulation of the present invention may be used to treat any conditions for which the agents to be delivered may be useful.

In another aspect of the invention, a method is provided for increasing the flux of a drug through mammalian dermal or mucosal surfaces. The method comprises applying to a mammalian subject a formulation comprising an active agent and a plant extract of the genus Curcuma or a natural or synthetic constituent thereof in an amount sufficient to enhance permeation of the active agent through mammalian dermal or mucosal surfaces.

Yet another embodiment of the present invention is a system for the delivery of active agents through mammalian dermal or mucosal surfaces. This system comprises a primary permeation enhancer comprising a plant extract of genus Curcuma or a natural or synthetic constituent thereof; and a secondary permeation enhancer wherein the permeation enhancers are present in a combined amount sufficient to enhance permeation of one or more active agents through mammalian dermal or mucosal surfaces. Useful secondary permeation enhancers include but are not limited to: an aliphatic alcohol, a polyalcohol, a monoalkyl ether of diethylene glycol, a tetraglycol furol or a mixture thereof. A most preferred secondary permeation enhancer is a mixture of water, a C₂ to C₄ alcohol, a polyalcohol, and either a monoalkyl ether of diethylene glycol or a tetraglycol furol.

In yet another embodiment of the invention, a pharmaceutical formulation is provided which comprises a therapeutically effective amount of an active agent, and a permeation enhancer comprising germacrone or a derivative thereof. The germacrone may be naturally or synthetically produced. In this regard, the germacrone may be synthesized by intramolecular alkylation of protected cyanohydin as described in T. Takahashi et al., Tetrahedron Letters, Vol. 24, No. 33, pp. 3489-3492 (1983), the contents of which are incorporated herein in its entirety by reference thereto. Additionally, germacrone can be extracted from C. zedoaria as described in M. Ohshiro et al., Phytochemistry, Vol. 29, No. 7, pp. 2201-2205 (1990), and from Smyrnium olustratum L. as described in A. Bertoli, Flavour and Fragrance Journal, Vol. 19, pp. 522-525 (2004), each of the contents of which are incorporated herein in their entirety by reference thereto. For the synthesis or extraction of germacrone, see also Phytochemisry, Vol. 31, No. 1, pp. 143-147 (1992), the content of which is incorporated herein in its entirety by reference thereto.

In accordance with the invention, the germacrone can be extracted from a plethora of plants including for the purpose of illustration and not limitation, plants of the genus Curcuma, rhododendron dauricum, thymus vulgaris, ledum groenlandicu, geranium macrorrhizum, citrullus aromatica and myrica gale.

In one aspect of the invention, the pharmaceutical formulation further includes a delivery vehicle comprising at least one of a monoalkyl ether of diethylene glycol, a polyalcohol, an alkanol, a tetraglycol furol or water.

In one embodiment, germacrone is present in an amount between about 0.10% to about 15% of the formulation by weight.

In another embodiment, the weight ratio of the germacrone to active agent is about 20:1 to about 1:20.

The active agent may be for example

It is to be understood that both the foregoing general description and the following detailed description are exemplary and are intended to provide further explanation of the invention claimed.

The accompanying figures, which are incorporated in and constitute part of this specification, is included to illustrate and provide a further understanding of the formulation, method and system of the invention. Together with the description, the figures serve to explain the principles of the invention.

### BRIEF DESCRIPTION OF THE FIGURES

FIG. 1 is a graph depicting transdermal absorption of oxybutynin over time in formulations including Curcuma zedoaria oil in an in vitro model;

FIG. 2 is a graph depicting the drug flux over time for oxybutynin in formulations including Curcuma zedoaria oil in an in vitro model;

FIG. 3 is a graph depicting transdermal absorption of progesterone over time in formulations including Curcuma zedoaria oil;

FIG. 4 is a graph depicting the drug flux over time for progesterone in formulations including Curcuma zedoaria oil in an in vitro model;

FIG. 5 is a graph depicting transdermal absorption of an anti-inflammatory drug, diclofenac diethylamonium, in formulations including Curcuma zedoaria oil in a in vitro model;

FIG. 6 is a graph depicting the drug flux over time for an anti-inflammatory drug, diclofenac diethylamonium, in formulations including Curcuma zedoaria oil in a in vitro model;

FIG. 7 is a graph depicting results of a comparative study of relative transdermal absorption of diclofenac diethylamonium with Curcuma zedoaria oil, ginger oil, and cinnamon oil;

FIG. 8 is a graph depicting transdermal absorption of progesterone over time of progesterone in formulations including Curcuma zedoaria oil in the presence of propylene glycol and diethylene glycol monoethyl ether;

FIG. 9 is a graph illustrating the comparative permeation enhancing effect of Curcuma zedoaria and Curcuma longa, both of which are species of the genus Curcuma;

FIG. 10 is a graph depicting the drug flux over time for an anti-inflammatory drug, diclofenac diethylamonium, in formulations including Curcuma zedoaria oil and Curcuma longa oil in an in vitro model;

FIG. 11 is a graph depicting results of a comparative study of transdermal absorption of diclofenac diethyamine over time in formulations including germacrone to a formulation without germacrone;

FIG. 12 graph depicting the drug flux over time for diclofenac diethylamine in formulations including germacrone as compared to a formulation without germacrone;

FIG. 13 is a graph illustrating results of a comparative study of the effect of germacrone on transdermal absoprtion of diclofenac diethylammonium; and

FIG. 14 is a graph depicting results of a comparative study of the effect of germacrone on the drug flux of diclofenac diethylammonium.

### DETAILED DESCRIPTION OF THE PREFERRED EMBODIMENTS

Reference will now be made in detail to the preferred embodiments of the invention, examples of which are illustrated in the accompanying figures.

The present invention relates generally to a formulation, system, and method for the topical, transdermal or transmucosal administration of active agents to a mammalian subject. The formulation comprises a therapeutically effective amount of an active agent, and a permeation enhancer comprising a plant extract of the genus Curcuma or natural or synthetic constituent thereof in an amount sufficient to enhance permeation of the active agent through mammalian dermal or mucosal surfaces. In another aspect of the invention, the formulation comprises a therapeutically effective amount of an active agent, and a permeation enhancer comprising germacrone or a derivative thereof.

The term “active agent” is used herein to refer to a compound or formulation or combination of compounds or formulations of matter which, when administered to an organism (human or animal) induces a desired pharmacological and/or physiologic effect by local and/or systemic action. The phrase “therapeutically effective amount” refers to a nontoxic but sufficient amount of a substance to provide the desired therapeutic effect.

Advantageously, it has been found that both the plant extract of Curcuma or a natural or synthetic constituent thereof, and germacrone or a derivative thereof, enhances the penetration of the active agent across the skin or mucosal barrier, the absorption rate of the active agent and induces the steady-state flux of the active agent to be reached in a relatively short time.

The phrase “genus of Curcuma” as used herein refers to any species or constituent of the Curcuma plant, including germacrone. The term “constituent” as used herein is meant to include any compound or substance of the Curcuma plant, including without limitation any compound or substance of the genus Curcuma as well as any of its species.

For the purpose of illustration, the species of the genus Curcuma can be selected from the group including but not limited to: Curcuma zedoaria, Curcuma alismatifolia, Curcuma amada, Curcuma angustifolia, Curcuma aromatica, Curcuma cordata, Curcuma petiolata, Curcuma elata, Curcuma flaviflora, Curcuma gracillima, Curcuma harmandii, Curcuma longa, Curcuma ornata, Curcuma roscoeana, Curcuma sparganifola, and Curcuma thorelii or any combination thereof. Preferably, the species is Curcuma Zedoaria. Preferably, the Curcuma zedoaria is present in the form of an oil. Preferably, the Curcuma zedoaria oil is present in an amount between about 0.1% to 15%; more preferably between about 0.5% to 10%, and most preferably between about 1% to 5% of the formulation.

The constituents extracted from the genus Curcuma, for example but not limitation, can be come from Curcuma zedoaria species and can be selected from the group including zedoarofuran, 4-epicurcumenol, neocurcumenol, gajutsulactones A and B, zedoarolides A and B, germacrone-4,5-epoxide, germacrone, furanodienone, curzerenone, zedereon, dehydrocurdione, curcumenol, isocurcumenol, curcumenone, curmanolide A, and curmanolide B or any combination thereof. Alternatively, the formulation may comprise constituents extracted from the genus Curcuma, other than Curcuma zedoaria or in addition to Curcuma zedoaria.

Alternatively, other constituents of the genus Curcuma, which are not explicitly referenced herein, as will be known in the art, are also in accordance with the present invention.

With respect to the formulation including a permeation enhancer comprising germacrone or a derivative thereof, the germacrone may be extracted from the genus Curcuma as discussed above, or alternatively, the germacrone or its derivatives may be synthesized or extracted from other plants as discussed supra.

The plant extract of the genus Curcuma or constituents of the genus Curcuma, such as Curcuma zedoaria can be extracted from the rhizome of Curcuma Zedoaria Roscoe plant or any other part of the plant such as the leaf, root, bulb, stem, flower, bark, inflorescence and seed.

General extraction methods, such as hydrodistillation (also known as Clevenger distillation) or steam distillation are well known in the art. Generally, Clevenger distillation relates to mixing the plant material to be extracted and water in the same reaction vessel, and boiling the mixture to evaporate both the water and the extract. Steam distillation generally relates to generating steam in a vessel that is separate from the reaction vessel, which contains the plant material. The steam is then fed into the bottom of the reaction vessel containing the plant material and the steam passes through plant material and volatizes the extract oils. In either method, the vapors of the volatile components and the steam are transported to a condenser where the condensation produces liquid having an oily layer and a water layer. The two layers can often be separated by simple decantation methods.

Specifically, the extraction of oils of the genus Curcuma is well known. For example, U.S. Pat. No. 6,344,5785, which is incorporated herein by reference thereto, discloses a process for extracting the oils from turmeric (Curcuma spp.). Accordingly, fresh rhizomes were distilled by a process including mechanically working the fresh plant material, boiling the worked plant material, collecting the distillate, and separating the components of the distillate to harvest the oils. Further, U.S. Pat. No. 5,176,913 to Honerlagen et al., which is incorporated herein by reference thereto, discloses a process for preparing concentrates of partial extracts of Curcuma zedoaria and further sub-species (Rhizoma). The Honerlagen process generally includes mixing the plant with an organic solvent, subjecting the extract to steam distillation, dehydrating the extract before evaporation, and distilling the organic solvent and recovering the oil concentrate.

In accordance with another aspect of the invention, a formulation is provided for topical, transdermal or transmucosal administration of an active agent comprising germacrone. It has been found that germacrone enhances permeation of an active agent across transdermal or transmucosal surfaces. For the purpose of illustration, germacrone can be extracted from various plants including Rhododendron dauricum, Thymus vulgaris, Ledum groenlandicum, Geranium macrorrhizum, Citrullus aromatica, Myrica gale, Smyrnium olusatrum and plants of the genus Curcuma.

Germacrone also named trans,trans-3,7-Dimethyl-10-isopropylidene-3,7-cyclodecadien-1-one is a sesquiterpene occurring in the essential oil of many plants. Examples of extractions of Germacrone are well disclosed in Flavour and Fragrance Journal, 2004; 19: 522-525, the content of which is incorporated herein in its entirety by reference. Germacrone has also been isolated by means of gas liquid chromatography-mass spectrometry from Zedoaria rhizoma essential oil as reported by Shibuya in Yakugaku Zasshi; 1986 March; 106(3):212-6, the content of which is incorporated by reference. Additionally, Germacrone can be obtained by synthesis by intramolecular alkylation of protected cyanohydrin. This total synthesis of Germacrone is well described by Takahashi in Tetrahedron Letters, Vol. 24, No. 33, pp 3489-3492, 1983, the entire contents of which is incorporated herein by reference.

The term “essential oil” refers to any of various volatile organic oils present in plants, usually containing terpenes and esters and having the odor or flavor of the plant from which the oil is extracted.

The formulations of the invention may include at least one or a combination of active agents. The “active agent” is used herein to refer to a substance or formulation or combination of substances or formulations of matter which, when administered to an organism (human or animal) induces a desired pharmacologic and/or physiologic effect by local and/or systemic action. The active agent administered may be any compound that induces a desired local or systemic effect and/or is suitable for topical, transdermal or transmucosal delivery.

The active agent may include the broad classes of compounds normally delivered through body surfaces and membranes, including the skin surface. In general, the classes of compounds include but are not limited to: analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; anti-inflammatory agents; antimigraine preparations; antinauseants; antineoplastic agents; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, CNS agents; beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; parasympatholytics; peptide drugs; psychostimulants; sedatives; steroids; sympathomimetics; tranquilizers; and vasodilators including general coronary, peripheral and cerebral.

Moreover, the active agent of the formulation may include any one of or a combination of steroid or nonsteroid hormones, their precursors, derivatives and analogs, esters and salts thereof including, but not limited to: dehydroepiandosterone (DHEA), androgens, estrogens and progestins (also referred to as progestogens).

Examples of androgens which may be used in this invention include testosterone (17-β-hydroxyandrostenone), and testosterone esters, such as testosterone enanthate, testosterone propionate and testosterone cypionate. The aforementioned testosterone esters are commercially available or may be readily prepared using techniques known to those skilled in the art or described in the pertinent literature. Also, pharmaceutically acceptable esters of testosterone and 4-dihydrotestosterone, typically esters formed from the hydroxyl group present at the C-17 position (such as enanthate, propionate, cypionate, phenylacetate, acetate, isobutyrate, buciclate, heptanoate, decanoate, undecanoate, caprate and isocaprate esters); and pharmaceutically acceptable derivatives of testosterone such as methyl testosterone, testolactone, oxymetholone and fluoxymesterone may be used.

Other suitable androgenic agents that may be used in the formulations of the present invention include, but are not limited to: the endogenous androgens, precursors and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, sodium dehydroepiandrosterone sulfate, 4-dihydrotestosterone (DHT), 5 adihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol.

Examples of estrogens and progestogens which may be useful in this invention include estrogens such as 17 beta-Estradiol, Estradiol, Estradiol Benzoate, Estradiol 17 beta-Cypionate, Estriol, Estrone, Ethynil Estradiol, Mestranol, Moxestrol, Mytatrienediol, Polyestradiol Phosphate, Quinestradiol, Quinestrol; progestogens such as Allylestrenol, Anagestone, Chlornadinone Acetate, Delmadinone Acetate, Demegestone, Desogestrel, Dimethisterone, Dydrogesterone, Ethynilestrenol, Ethisterone, Ethynodiol, Ethynodiol Diacetate, Flurogestone Acetate, Gestodene, Gestonorone Caproate, Haloprogesterone, 17-Hydroxy-16-methylene-progesterone, 17 alpha-Hydroxyprogesterone, 17 alpha-Hydroxygesterone Caproate, Lynestrenol, Medrogestone, Medroxyprogesterone, Megestrol Acetate, Melengestrol, Norethindrone, Norethindrone Acetate, Norethynodrel, Norgesterone, Norgestimate, Norgestrel, Norgestrienone, 19-Norprogesterone, Norvinisterone, Pentagestrone, Progesterone, Natural Progesterone, Promegestone, Quingestrone, Trengestone.

Other suitable active agents include but are not limited to anti estrogens, such as Tamoxifen, 4-OH Tamoxifen; anti progestogens and anti androgens, alpha-Adrenergic Agonists, such as Budralazine, Clonidine, Epinephrine, Fenoxazoline, Naphazoline, Phenylephrine, Phenylpropanolamine, beta-Adrenergic Agonists such as Formoterol, Methoxyphenamine, alpha-Adrenergic Blockers such as Doxazosin, Prazosin, Terazosin, Trimazosin, Yohimbine, beta-Adrenergic Blockers such as Atenolol, Bisoprolol, Carteolol, Carvedilol, Metoprolol, Nadolol, Penbutolol, Analgesics (Narcotics or Non-Narcotics) such as Buprenorphine, Dihydromorphine, Metazocine, Methadone, Morphine, Morphine Derivatives, Nicomorphine, Oxymorphone.

Other suitable active agents include sedatives and anxyolitics for instance Benzodiazepine derivatives such as Alprazolam, Bromazepam, Flutazolam, Ketazolam, Lorazepam, Prazepam; Amides such as Butoctamide, Diethylbromoacetamide, Ibrotamide, Isovaleryl Diethylamide, Niaprazine, Tricetamide, Trimetozine, Zolpidem, Zopiclone; Arylpiperazines such as Buspirone.

Other suitable active agents include nerve agents for smoking cessation, such as Nicotine, Nicotine Citrate and Nicotine Tartrate; Antineoplastic Agents such as 5-Fluorouracil; Anti-Inflammatory Agents, such as diclofenac; Anesthetics; Antianginals; Anticholinergics such as oxybutynin; Anticonvulsants; Antidepressants; Antiepileptics; Antiestrogen; Antihistaminics; Antiparkinsonians; Bronchodilators; Diuretics; Glucocorticoids; Muscle Relaxants; Narcotic Antagonists; Antihypothyroids such as Levothyroxine, Thyroid, Thyroxine; Antihypertensives for instance Benzothiadiazine Derivatives such as Captopril, Cilazapril, Enalapril, Lisinopril, Perindopril, Ramipril; Guanidine Derivatives such as Guanethidine; Quinazoline Derivatives such as Alfuzosin; Reserpine Derivatives such as Reserpine, Sulfonamide Derivatives such as Furosemide; others such as Minoxidil, Amlodipine, Terbinafine, Doxazosin Mesylate, Felodipine, Moxonidine, Nicardipine Hydrochloride, Nifedipine, Prazosin hydrochloride, etc and Calcium Channel Blockers such as Arylalkylamines such as Bepridil, Ditiazem, Fendiline, Gallopamil, Terodiline, Verapamil; Dihydropyridine Derivatives such as Felodipine, Isradipine, Nicardipine, Nifedipine, Nilvadipine, Nimodipine, Nisoldipine, Nitrendipine, piperazine; Derivatives such as Flunarisine; others such as Perhexiline; Calcium Regulator such as Calcifediol, Calcitonin, Calcitriol, Clodronic Acid, Dihydrotachysterol, Elcatonin, Etidronic Acid, Ipriflavone, Pamidronic Acid, Parathyroid Hormone, Teriparatide Acetate.

In a preferred embodiment, the weight ratio of permeation enhancer to active agent is between about 3:0.5 to 0.5:3.

Preferably, the weight percent ratio of C. zedoaria oil to active agent is between about 10:1 to 1:10, preferably 6:1 to 1:6, and more preferably from 3:1 to 1:3.

Preferably, the weight percent ratio of germacrone to active agent is between about 20:1 to 1:20, preferably 10:1 to 1:10, and more preferably from 5:1 to 1:5.

In one embodiment of the invention, the formulation further comprises a delivery vehicle or carrier. The terms “carrier” or “vehicle” as used herein refer to materials suitable for transdermal drug administration, and include any such material known in the art, including any liquid, gel, solvent, liquid diluent, solubilizer or the like, which is nontoxic and which does not interact with other components of the composition in a deleterious manner.

The delivery vehicle comprises at least one of a C₂ to C₄ alcohol, a polyalcohol, a monoalkyl ether of diethylene glycol, a tetraglycol furol, or water. Preferably, the concentration of permeation enhancer is between about 0.05 and 15% w/w. Preferably, the concentration of polyalcohol is between about 1.0 and 30% w/w. Preferably, the concentration of C2 to C4 alkanol is between about 5 and 75% w/w. Preferably, the concentration of water is between about 15 and 80% w/w.

For purpose of illustration the C₂ to C₄ alkanol may include ethanol, isopropanol, n-propanol, butanol, and preferably ethanol. The polyalcohol may include propylene glycol, butylene glycol, hexylene glycol, and ethylene glycol. Preferably, the polyalcohol is propylene glycol. The preferred monoalkyl ether of diethylene glycol is diethylene glycol monoethyl ether or diethylene glycol monomethyl. Most preferably, diethylene glycol monoethyl ether is used The tetraglycol furol is preferably glycofurol having the formula: (C₂H₄O)mult-C₅—H₁₀—O₂, and is represented by the structural formula:

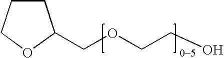

The formulation may further include a thickening agent or gelling agent present in an amount sufficient to alter the viscosity of the formulation. A gelling agent can be selected from the group including: carboxyethylene, acrylate copolymers, sodium polyacrylates, acrylic acid and alkyl methacrylate copolymers or polyacrylic acid such as Carbopol 980 or 940 NF, 981 or 941 NF, 1382 or 1342 NF, 5984 or 934 NF, ETD 2020, 2050, 934P NF, 971P NF, 974P NF, Carbopol Ultrez 10, Noveon AA-1 USP, Avalure AC and UR, Pemulen TR; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel different grades), hydroxyethylcellulose (HEC) (Natrosol grades), HPMCP 55, Methocel grades; natural gums such as arabic, xanthan, guar gums, alginates; polyvinylpyrrolidone derivatives such as Kollidon grades; polyoxyethylene polyoxypropylene copolymers such as Lutrol F grades 68, 127. Other gelling agents include chitosan, polyvinyl alcohols, polyethylene glycols, dextranes, silicones, carrageen, silica, pectines, veegum grades, methylvinyl ether-maleic acid anhydride copolymers, polyoxyethylene-polyoxypropylene block polymers, polyacrylates, polyacrylamides and polyvinyl alcohols and acetates. Preferably, the gelling agent is cellulose and polyacrylic acid. Alternatively, other gelling agents known by those skilled in the art may also be used. The preferred gelling agent is present from about 0.1% to about 10% w/w depending on the type of polymer.

The formulation may further include preservatives such as but not limited to benzalkonium chloride and derivatives, benzoic acid, benzyl alcohol and derivatives, bronopol, parabens, cetrimide, chlorhexidine, cresol and derivatives, imidurea, phenol, phenoxyethanol, phenylethyl alcohol, phenylmercuric salts, thimerosal, sorbic acid and derivatives. Alternatively, other preservatives known by those skilled in the art may also be used. The preservative is present from about 0.01 to about 10% w/w depending on the type of compound.

The formulation may optionally include antioxidants such as but not limited to tocopherol and derivatives, ascorbic acid and derivatives, butylated hydroxyanisole, butylated hydroxytoluene, fumaric acid, malic acid, propyl gallate, metabisulfates and derivatives. The antioxidant is present from about 0.001 to about 5% w/w depending on the type of compound. Alternatively, other antioxidants known by those skilled in the art may be also be used.

The formulation may further include buffers such as carbonate buffers, citrate buffers, phosphate buffers, acetate buffers, hydrochloric acid, lactic acid, tartric acid, diethylamine, triethylamine, diisopropylamine, aminomethylamine. Alternatively, other buffers known by those skilled in the art may also be used. The buffer may replace up to 100% of the water amount within the formulation.

The formulation may further include humectants, such as but not limited to glycerin, propylene, glycol, sorbitol, triacetin, urea and urea derivatives. Alternatively, other humectants known by those skilled in the art may also be used. The humectant is present from about 1 to 30% w/w depending on the type of compound.

The formulation may further include a sequestering agent such as edetic acid. Alternatively, other sequestering agents known by those skilled in the art may also be used. The sequestering agent is present from about 0.001 to about 5% w/w depending on the type of compound.

The formulation may further include anionic, non-ionic or cationic surfactants. Alternatively, other surfactants known by those skilled in the art may also be used. The surfactant is present from about 0.1% to about 30% w/w depending on the type of compound.

Optionally, the formulation may include a pH regulator, generally, a neutralizing agent, which can optionally have crosslinking function. By way of example and not limitation, the pH regulator may include an amine such as diethanolamine, diethylamine, ethanolamine, monoethanolamine, triethanolamine, trolamine, tromethamine, tetrahydroxypropylethylendiamine, diisopropanolamine, or chlorhydric acid or sodium hydroxide solution. The pH regulator is present in the formulations in about 0.05 to about 5.0% w/w.

The formulation may further include solvents including but not limited to polyethylene glycol, N-methylpyrrolidone, 2-pyrol, dimethylisosorbide, as well as other solvents known in the art.

In accordance with another aspect of the invention, a system is provided for the delivery of active agents through mammalian dermal or mucosal surfaces. The system of the invention comprises a primary permeation enhancer comprising a plant extract of genus Curcuma or a natural or synthetic constituent thereof, and a secondary permeation enhancer. The primary and the secondary permeation enhancers are present in a combined amount sufficient to enhance permeation of one or more active agents through mammalian dermal or mucosal surfaces.

In one embodiment, the system may include a secondary permeation enhancer comprises at least one of a C₂ to C₄ alcohol, an aliphatic alcohol, a polyalcohol, a monoalkyl ether of diethylene glycol, a tetraglycol furol, water or any mixture thereof.

In another aspect of the invention, a method is provided for increasing the flux of a drug through mammalian dermal or mucosal surfaces. The method comprises applying to a mammalian subject in need of the active agent a formulation comprising a therapeutically effective amount of an active agent, and a permeation enhancer comprising a plant extract of genus Curcuma or a natural or synthetic constituent thereof in an amount sufficient to enhance permeation of the active agent through mammalian dermal or mucosal surfaces.

The formulation may be in an adapted form for topical, transdermal or transmucosal administration. For example, the formulation can be in the form of a gel, spray, ointment, aerosol, patch, foam, buccal and sublingual tablets, suppositories, vaginal dosage forms, or other passive or active transdermal devices for absorption through the skin or mucosal surface, including those of the oral cavity. In accordance with the invention, the transdermal formulation may be in the form of a transdermal patch comprising an adhesive layer or matrix comprising the formulation of the invention, a backing layer that is impermeable to the formulation and adhesive, and a protective liner releasably attached to the adhesive layer such that the formulation is covered by the liner and unexposed until the protective liner is peeled off by the patch user. Typically, the patch adhesive layer or matrix serves as the carrier for the active agent or active agents to be administered to the patch user. Alternatively, additional layers may be included between the patch adhesive or matrix layer and the backing layer to include additional active agents, or non-toxic polymers well known in the art used to carry drugs or act as rate-controlling membranes.

The following examples of the formulations of the present inventions are for the purpose of illustration and not limitation.

### EXAMPLES

All the quantities have been expressed in percentage weight by weight (% w/w).

### Example 1

A reference gel containing oxybutynin base 3.00%, ethanol 50.0%, propylene glycol 15.0%, diethylene glycol monoethylether (TRANSCUTOL™ P from GATTEFOSSE) 2.50%, hydroxypropylcellulose (KLUCEL HF Pharm™ from HERCULES) 2.00%, butyl hydroxy toluene 0.05%, urea 5.00%, hydrochloride solution 0.1M qs pH 7.0-7.5 and purified water qs 100% was prepared by dissolving the active ingredient (if not hydrosoluble) in the ethanol/propylene glycol/diethylene glycol monoethylether. Hydrochloride solution 0.1M was added in previous alcoholic solution to adjust the pH between 7.00-7.50. The purified water was then added and hydroxypropylcellulose thoroughly dispersed in the hydro-alcoholic solution under mechanical stirring at room temperature at a suitable speed ensuring good homogenization of the formulation while avoiding lumps formation and air entrapment.

### Example 2

A gel containing oxybutynin base 3.00%, ethanol 33.5%, propylene glycol 15.0%, diethylene glycol monoethylether (TRANSCUTOL™ P) 2.50%, hydroxypropylcellulose (KLUCEL HF Pharm™) 2.00%, butyl hydroxy toluene 0.05%, essential oils combination containing zedoary oil at an undisclosed concentration (ZEDOMINE™ from VEVY EUROPE S.P.A.) 1.00%, isopropanol 20.0%, hydrochloride solution 0.1M qs pH 7.0-7.5 and purified water qs 100% was prepared as described in example 1. Essential oils mixture was added in alcoholic phase.

### Example 3

A reference gel containing progesterone 2.00%, diethylene glycol monoethylether (TRANSCUTOL™ P) 5.00%, propylene glycol 6.00%, isopropanol 60.0%, purified water 25.0% and hydropropylcellulose (KLUCEL HF Pharm™) 2.00% was prepared by dissolving the active ingredient (if not hydrosoluble) in the isopropanol/propylene glycol/diethylene glycol monoethylether. The purified water was then added and hydroxypropylcellulose thoroughly dispersed in the hydro-alcoholic solution under mechanical stirring at room temperature at a suitable speed ensuring good homogenization of the formulation while avoiding lumps formation and air entrapment.

### Example 4

A gel containing progesterone 2.00%, diethylene glycol monoethylether (TRANSCUTOL™ P) 5.00%, propylene glycol 6.00%, isopropanol 60.0%, purified water 24.0%, hydropropylcellulose (KLUCEL HF Pharm™) 2.00% and ZEDOMINE™ 1.00% was prepared as described in example 3. Essential oils mixture was added in alcoholic phase.

### Example 5

A gel containing progesterone 2.00%, diethylene glycol monoethylether (TRANSCUTOL™ P) 5.00%, propylene glycol 6.00%, isopropanol 60.0%, purified water 22.0%, hydropropylcellulose (KLUCEL HF Pharm™) 2.00% and ZEDOMINE™ 3.00% was prepared as described in example 3.

### Example 6

A reference gel containing diclofenac diethylammonium 1.16%, diethylene glycol monoethyl ether (TRANSCUTOL™ P) 5.00%, propylene glycol 6.00%, isopropanol 60.0%, purified water 24.84% and hydroxypropylcellulose (KLUCEL HF Pharm™) 2.00% was prepared as described in example 3.

### Example 7

A gel containing diclofenac diethylammonium 1.16%, diethylene glycol monoethyl ether (TRANSCUTOL™ P) 5.00%, propylene glycol 6.00%, isopropanol 60.0%, purified water 24.84%, hydroxypropylcellulose (KLUCEL HF Pharm™) 2.00% and ZEDOMINE™ 1.00% was prepared as described in example 3.

### Example 8

A gel containing diclofenac diethylammonium 1.16%, diethylene glycol monoethyl ether (TRANSCUTOL™ P) 5.00%, propylene glycol 6.00%, isopropanol 60.0%, purified water 22.84%, hydroxypropylcellulose (KLUCEL HF Pharm™) 2.00% and ZEDOMINE™ 3.00% was prepared as described in example 3.

### Example 9

A gel containing diclofenac diethylammonium 1.16%, diethylene glycol monoethyl ether (TRANSCUTOL™ P) 5.00%, propylene glycol 6.00%, isopropanol 60.0%, Curcuma zedoaria OIL (from INDO WORLD TRADING CORPORATION) 1.00%, purified water 24.84% and hydroxypropylcellulose (KLUCEL HF Pharm™) 2.00% was prepared as described in example 3.

### Example 10

A gel containing diclofenac diethylammonium 1.16%, diethylene glycol monoethyl ether (TRANSCUTOL™ P) 5.00%, propylene glycol 6.00%, isopropanol 60.0%, Zingiber officinale OIL (from INDO WORLD TRADING CORPORATION) 1.00%, purified water 24.84% and hydroxypropylcellulose (KLUCEL HF Pharm™) 2.00% was prepared as described in example 3.

### Example 11

A gel containing diclofenac diethylammonium 1.16%, diethylene glycol monoethyl ether (TRANSCUTOL™ P) 5.00%, propylene glycol 6.00%, isopropanol 60.0%, Cinnamon zeylanicum OIL (from INDO WORLD TRADING CORPORATION) 1.00%, purified water 24.84% and hydroxypropylcellulose (KLUCEL HF Pharm™) 2.00% was prepared as described in example 3.

### Example 12

A reference gel containing progesterone 2.00%, isopropanol 60.0%, purified water 35.4%, CARBOPOL™ 1382 (from NOVEON) 0.60% and triethanolamine 0.20% was prepared by dissolving the active ingredient in isopropanol. Purified water was then added and CARBOPOL™ 1382 was dispersed in the hydro-alcoholic solution under mechanical stirring at room temperature at a suitable speed ensuring good homogenization of the formulation while avoiding lumps formation and air entrapment. Triethanolamine was added in the end of the manufacturing process to neutralize the gel.

### Example 13

A gel containing progesterone 2.00%, Curcuma zedoaria OIL 1.00%, isopropanol 60.0%, purified water 36.2%, CARBOPOL™ 1382 0.60% and triethanolamine 0.20% was prepared as described in example 12. Zedoary oil was added to alcoholic phase.

### Example 14

A gel containing progesterone 2.00%, Curcuma zedoaria OIL 3.00%, isopropanol 60.0%, purified water 34.2%, CARBOPOL™ 1382 0.60% and triethanolamine 0.20% was prepared as described in example 12. Zedoary oil was added to alcoholic phase.

### Example 15

A gel containing diclofenac diethylammonium 1.16%, isopropanol 60.0%, diethylene glycol monoethyl ether (TRANSCUTOL™ P) 5.00%, propylene glycol 6.00%, Curcuma longa OIL (from INDO WORLD TRADING CORPORATION) 1.00%, purified water 24.84% and hydroxypropylcellulose (KLUCEL HF Pharm™) 2.00% was prepared as described in example 3.

Various illustrations of the present formulations and permeation enhancement of active agent across the dermal or transmucosal surfaces of a mammalian subject were carried out in vitro as described below.

In Vitro Comparative Studies

In vitro drug permeation and biodistribution experiments through ear pig skin were made using a Franz Vertical Diffusion Cell diffusion chamber. Cutaneous penetration studies in vitro through human skin are limited due to the lack of availability of the human skin. It is largely described in the literature that ear pig skin can be used as the closest model to human skin in the assessment of percutaneous absorption of chemicals.

In Vitro Permeation Experiments

Fresh cadaver ear pig skin obtained from slaughterhouses was processed according to standard operating procedures. The ears were evaluated on their integrity (no bites, scratches or redness) and condition. The skin was excised from the ears with the help of scalpels, avoiding perforations or any damage. The excised skin samples were rinsed with Phosphate Buffered Saline (PBS) solution and placed on a surface for successive punching of skin disks. The skin disk pieces were mounted between the sections of a vertical diffusion cell having 1.77 sqcm of surface area, the epidermal facing up. 50 mg of the transdermal devices exemplified previously was applied over the epidermal layer whilst the dermal layer contacts with the receptor solution: 2.0% w/V polyoxyethylene 20 oleyl ether (Oleth 20), with PBS solution, pH 7.4. The receptor chamber was maintained at 35° C. and the studies were conducted under non-occlusive conditions and at 600 rpm of stirring speed. At given time points, samples were withdrawn from the receptor solution and the receptor chamber was immediately refilled with fresh solution. All samples taken from the receptor solution (permeated drug) were analyzed using a high performance liquid chromatography (HPLC) method.

Cumulated Drug Permeated and Drug Flux Determination (In Vitro Permeation Study)

The total amount of drug permeated (mcg/cm2) and the transdermal flux (mcg/sqcm/h) were determined for each study.

Biodistribution Experiments

After completion of the permeation study, and utilizing appropriate solvents composition, all skin disk pieces were analyzed in drug distribution within the skin layers: dermis, epidermis and stratum corneum. Unabsorbed formulation was also assessed. Then, balance mass was performed in order to assess total recovery/distribution of drug after certain time following drug product administration/application, considering unabsorbed formulation, the amount of drug in the stratum corneum and the amount of drug within the innermost layers of the skin (epidermis, dermis, and receptor solution representing the bloodstream). The different compartments were analyzed using a high performance liquid chromatography (HPLC) method.

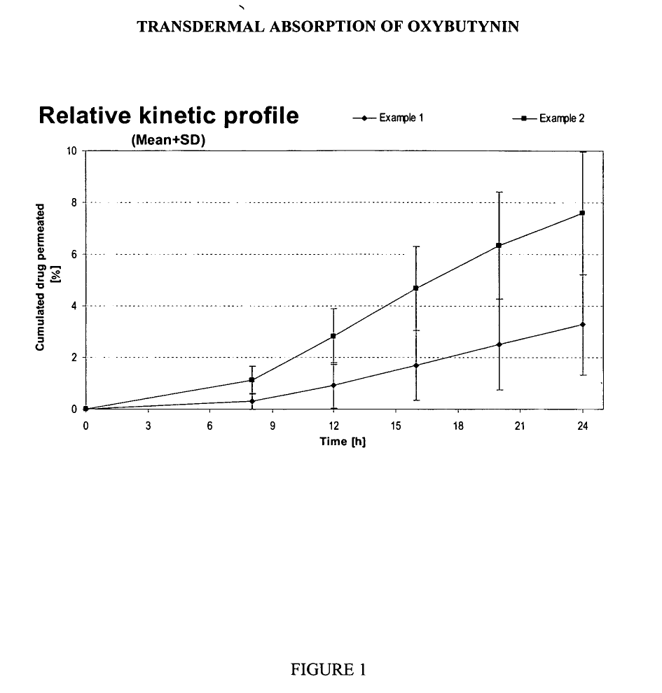

The accompanying figures represent studies which further exemplify the invention described herein. The figures are for the purpose of illustration and not for the limitation of the invention. With reference to FIG. 1 a graph illustrating the effects of Curcuma zedoaria oil used as a permeation enhancer for oxybutynin. A mixture of essential oils comprising zedoary oil, ginger oil, and cinnamon oil (ZEDOMINE™ from VEVY EUROPE S.P.A.) was tested in systemic drug delivery systems. In particular, a comparison study was performed of reference example 1, which contained 3.00% w/w oxybutynin and no C. zedoaria oil, example 2, which contained oxybutynin 3.00% and 1.00% ZEDOMINE™ containing an undisclosed amount of C. zedoaria oil (X %). The results of this study illustrate the transdermal absorption of oxybutynin for example 2, those with the C. zedoaria oil, were higher than the reference example 1 and are statistically significant from 16 h permeation and beyond. The daily absorbed doses were, respectively, 2.3 fold (13.44 μg/cm² versus 5.77 μg/cm²) higher for examples 2 than for example 1.

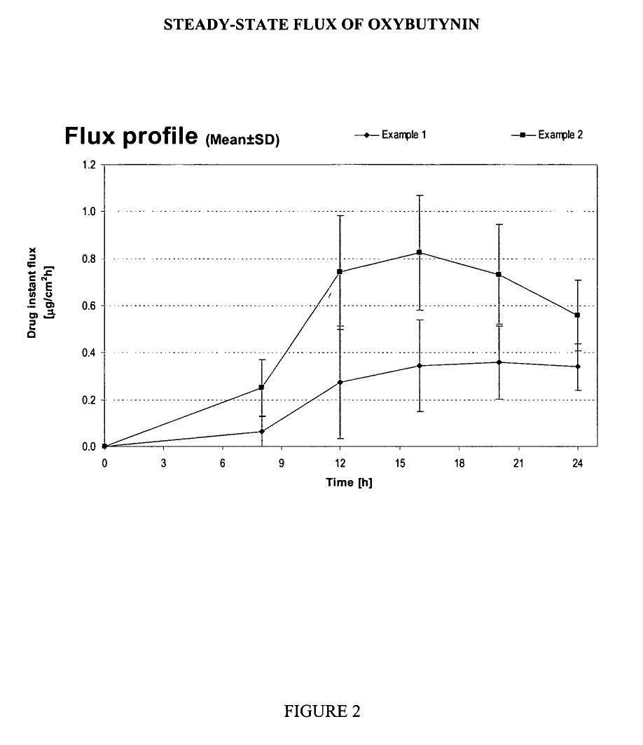

FIG. 2 is a graph illustrating an in vitro study illustrating the drug flux profile of examples 1, 2, described above. As shown, the steady-state fluxes were reached 4 h sooner for example 2, the formulation comprising the C. zedoaria oil, than for reference example 1, which did not contain C. zedoaria oil. Further, the steady-state fluxes for example 2 was 1.9 fold higher than the steady-state flux for example 1, respectively, 0.65 vs. 0.34 μg/cm² h. These studies show that ZEDOMINE™ significantly increases the transdermal penetration of oxybutynin.

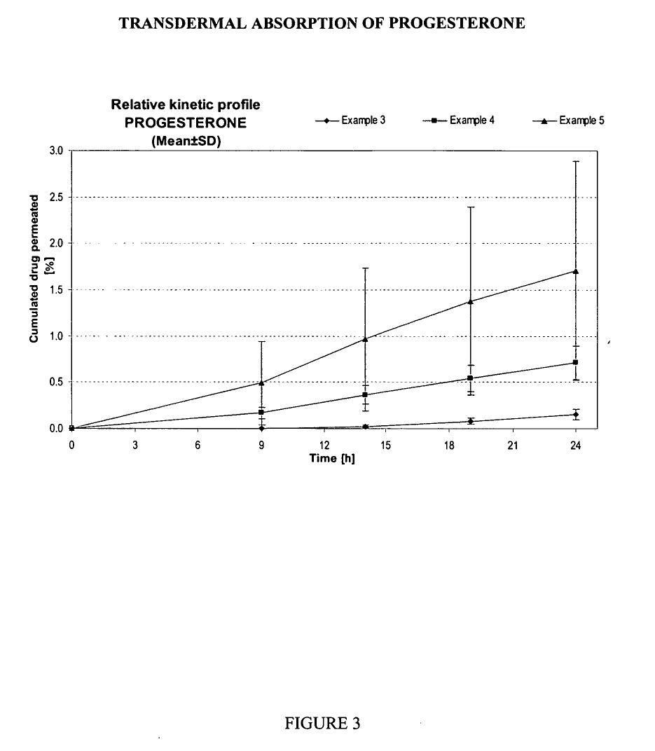

FIG. 3 is a graph illustrating an in vitro study illustrating the effects of C. zedoaria oil used as a permeation enhancer for progesterone. ZEDOMINE™ was tested in systemic drug delivery systems. In particular, a comparison study was performed of reference example 3, which contained 2.00% w/w progesterone and no C. zedoaria oil, example 4, which contained progesterone 2.00% and 1.00% ZEDOMINE™ containing an undisclosed amount of C. zedoaria oil (X %), and example 5, which contained 2.00% progesterone and 3.00% ZEDOMINE™ containing an undisclosed amount of C. zedoaria oil (3X %). The results of this study illustrate the transdermal absorption of progesterone for examples 4 and 5, those with the C. zedoaria oil, were higher than the reference example 3. For formulation 4, the enhancement ratio was 4.6, whereas for formulation 5, it is even higher; 10.9. These values are statistically different compared to the reference formulation without C. zedoaria oil, even if variability is higher for formulation 4 than 5.

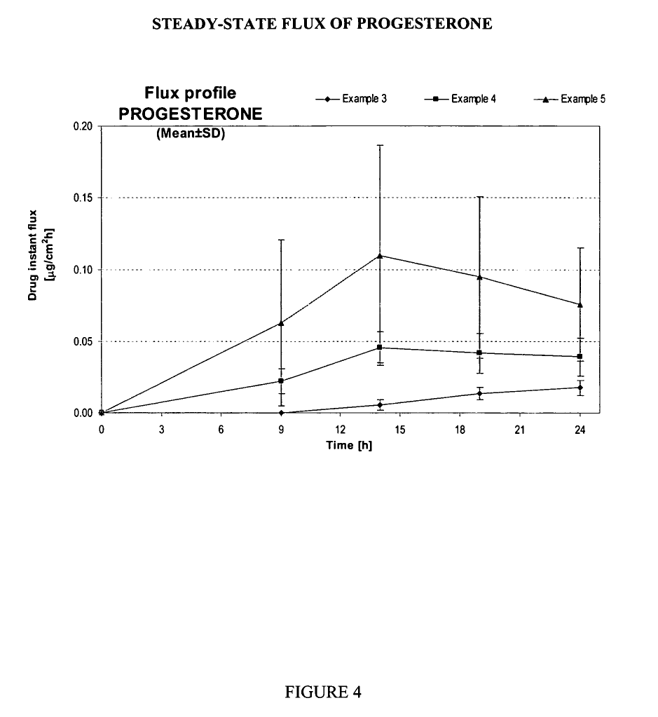

FIG. 4 is a graph depicting the drug flux of progesterone in examples 3, 4, and 5 described above. Steady-state was not attained for the reference formulation, whereas progesterone in the presence of ZEDOMINE™ is attained at 14 h. Thus, an additional advantage of the C. zedoaria oil is a more rapid onset action of the drug. Thus, studies illustrated in FIGS. 3 and 4 illustrate that the efficacy of ZEDOMINE™ which increases significantly the permeation of progesterone through the skin.

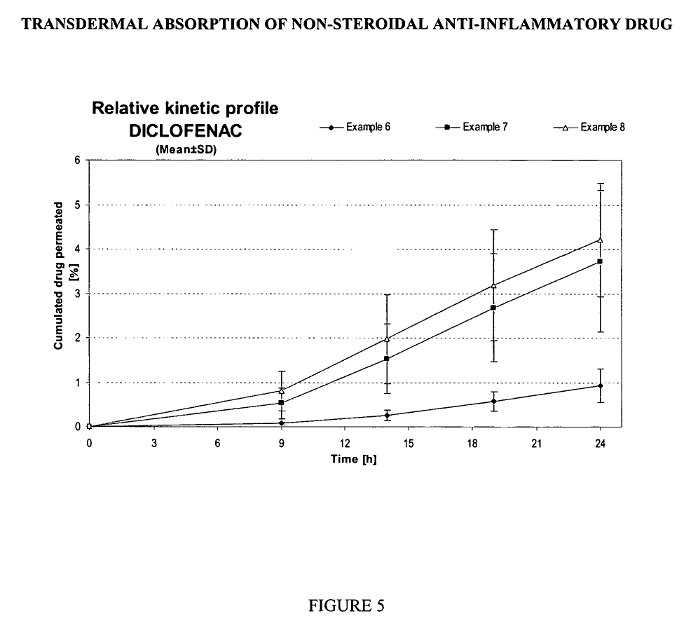

FIG. 5 is a graph illustrating an in vitro study illustrating the transdermal absorption of formulation examples 6, 7 and 8 containing anti-inflammatory drug, diclofenac diethylamonium. Reference example 6 contained 1.16% diclofenac diethylamonium with no C. zedoaria oil, example 7 contained 1.16% diclofenac diethylamonium and 1.00% ZEDOMINE™ containing an undisclosed amount of C. zedoaria oil (X %); and formulation 8 contained 1.16% diclofenac diethylamonium and 3.00% ZEDOMINE™ containing an undisclosed amount of C. zedoaria oil (3X %). The addition of ZEDOMINE™ in diclofenac diethylamonium formulations significantly increased the absorption of the drug through the skin. In particular, formulations 7 and 8 containing respectively 1% and 3% ZEDOMINE™ have drug absorption increase by about 4. The relative cumulated permeated amount is respectively, 0.925% for example 6, 3.730% for example 7, and 4.208% for example 8.

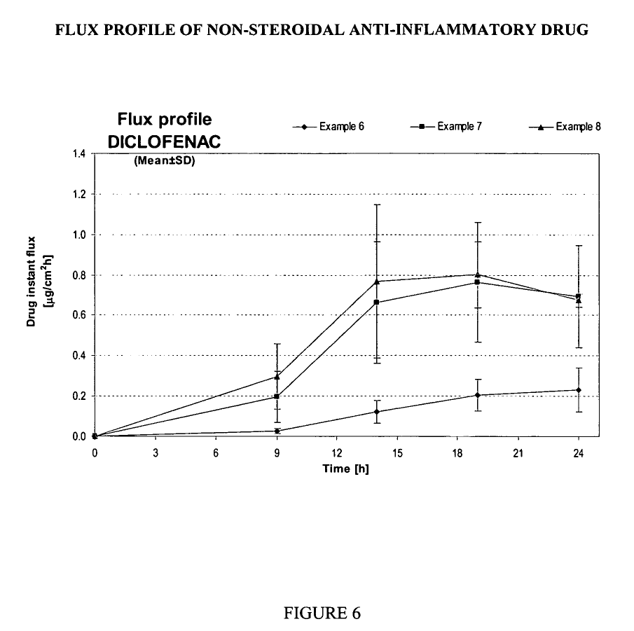

FIG. 6 is a graph illustrating the drug flux profile of formulation examples 6, 7, and 8, described above. As shown, the steady-state fluxes were reached 19 h sooner for the formulations containing the C. zedoaria oil.

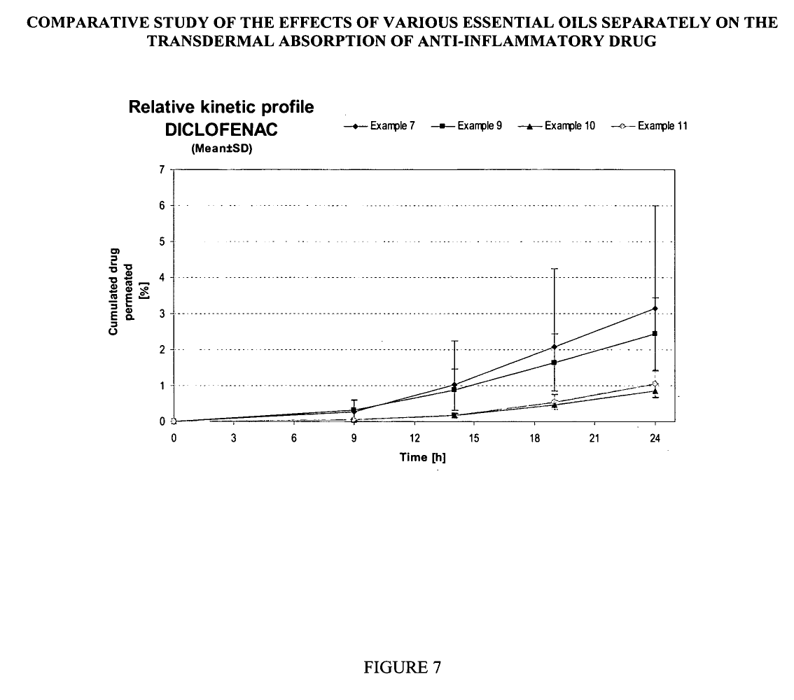

FIG. 7 is a graph depicting the effects of ZEDOMINE™ versus C. zedoaria oil, ginger oil, and cinnamon oil separately. For each of the formulations, the ZEDOMINE™ and oils were mixed with propylene glycol and diethylene glycol monoethylether. Formulation example 7 contained diclofenac diethylamonium 1.16% and 1.00% ZEDOMINE™, formulation example 9 contained diclofenac diethylamonium 1.16% and C. zedoaria oil 1.00%; formulation example 10 contained diclofenac diethylamonium 1.16% and ginger oil 1.00%, and formulation example 11 contained diclofenac diethylamonium and 1.00% cinnamon oil. The results show that C. zedoaria oil (example 9) is the main permeation enhancer of the active agent diclofenac through the skin compared to example 10, ginger oil, and example 11, cinnamon oil. The study illustrates that C. zedoaria oil accounts for 77% of the cumulated diclofenac permeated after 24 h compared to example 7, 8.01 μg/cm² for example 9 and 10.34 μg/cm² for example 7. The two other essential oils, ginger and cinnamon, are shown to have almost no effect on drug absorption. Both the ginger and cinnamon oils account for only about 30% compared to the example 7 containing the mixture of ginger, cinnamon, and C. zedoaria oils. These results illustrate the efficacy of C. zedoaria oil for increasing permeation of diclofenac through the skin. Zedoary oil exhibits the main effect over the two other essential oils present in ZEDOMINE™. This study also demonstrated that any essential oil is not efficient as a permeation enhancer.

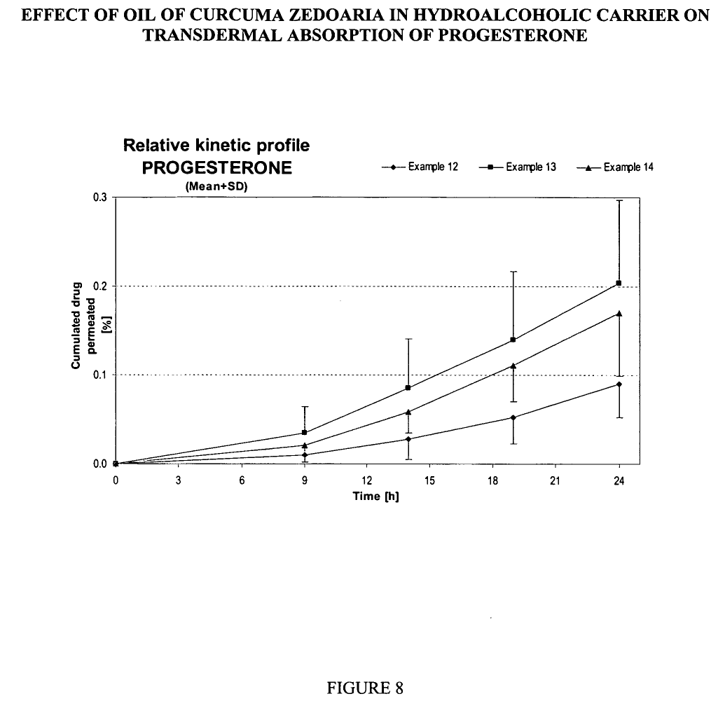

FIG. 8 is a graph depicting the effect of the addition of C. zedoaria oil in a simple hydro-alcoholic carrier in comparison with studies shown at FIG. 7. Thus, the graph depicts the effect of C. zedoaria oil at 1 and 3% in hydroalcoholic gels on the transdermal absorption of progesterone. Examples 12,13, and 14 each contained 2.00% progesterone. Additionally, examples 12, 13, and 14 contained, no C. zedoaria oil, 3.00% C. zedoaria oil, and 1.00% zedoaria oil, respectively. Each formulation was in the form of a hydroalcoholic gel. As shown, examples 13 and 14, which contained the C. zedoaria oil exhibit a 2.2 fold better relative transdermal absorption than the reference example without Zedoaria oil. In particular, 1.16 μg/cm² vs. 0.51 μg/cm² and 0.20% vs. 0.09%. As shown, the maximum steady-state flux is 1.8-fold higher for the formulations comprising C. zedoaria oil; 0.07 μg/cm²h vs. 0.04 μg/cm²h. Thus, without the addition of co-solvents or co-enhancers, C. zedoaria oil significantly increases absorption of progesterone across the skin barrier.

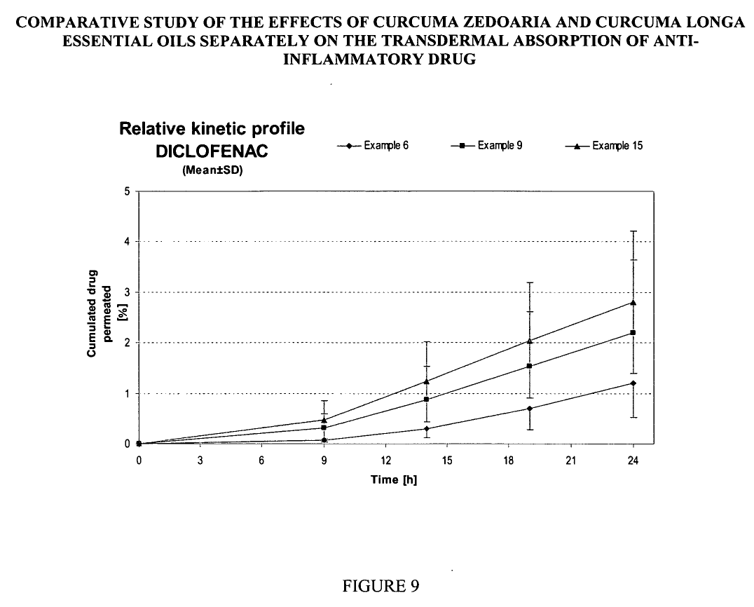

FIG. 9 is a graph depicting the effect of example 9 Curcuma zedoaria oil 1.00% in comparison with example 15 Curcuma longa oil 1.00% (other specie of genus Curcuma) on the transdermal absorption of diclofenac diethylamonium 1.16%. For each formulation, the oils were mixed with propylene glycol and diethylene glycol monoethyl ether.

The graph showed a clear enhancing effect of both Curcuma Zedoaria and Curcuma Longa oils on the systemic uptake of diclofenac diethylammonium. Compared to the reference, example 6, without essential oils, the tested formulations allow to increase diclofenac diethylamonium absorption by, respectively, 1.8 times (7.28 μg/c12 for example 9, against 3.97 μg/cm2 for example 6), and 2.3 times (9.25 μg/cm2 for example 15, against 3.97 μg/cm2 for example 6), even if differences are not statistically significant due to the large variability.

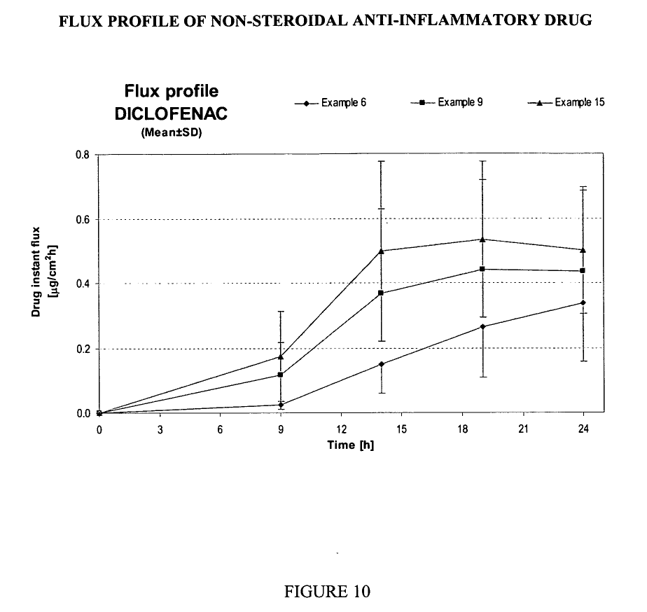

FIG. 10 is a graph illustrating the drug flux profile of formulation examples 6, 9, and 15 described above. Another interesting point is the shorter onset of steady-state for the formulations containing the essential oils, which is attained after 19 h permeation, in contrast to the reference, in which the steady-state onset was not yet attained after 24 h permeation.

The effects of germacrone on the permeation of diclofenac diethylammonium was investigated. In the study, a comparison was made of a gel containing diclofenac 1.16% and germacrone (Example 17 below), a gel comprising diclofenac and zedoaria oil (Example 16 below) and a marketed reference gel containing diclofenac diethylamine (VOLTARENE Emulgel 1%—Novartis Pharma S.A.).

| | VOLTARENE | Example | Example |
|---|---|---|---|
| Formulation | Emulgel 1% | 16 | 17 |
| Diclofenac diethyl ammonium | 1.16 | 1.16 | 1.16 |
| Propylene glycol | 5.00 | 6.00 | 6.00 |
| Ethoxydiglycol | — | 5.00 | 5.00 |
| (Transcutol ® P) | | | |
| Zedoaria oil | — | 1.00 | — |
| (Greentech S.A.) | | | |
| Germacrone (FLUKA Chemie | — | — | 0.50 |
| GmbH) | | | |
| Hydroxypropyl cellulose | — | 1.50 | 1.50 |
| (Klucel HF) | | | |
| Absolute ethanol | — | 50.00 | 50.00 |
| Diethylamine | 0.90 | — | — |
| Carbomer 974P | 1.20 | — | — |
| Macrogol 1000 | 2.00 | — | — |
| Monocetylether | | | |
| Cetiol LC | 2.50 | — | — |
| Isopropanol | 20.00 | — | — |
| Liquid Paraffin | 2.50 | — | — |
| Fragrance (Cream 45) | 0.10 | — | — |
| Purified Water | 64.64 | 35.34 | 35.84 |

Amounts are expressed as percent weight by weight (w/w).

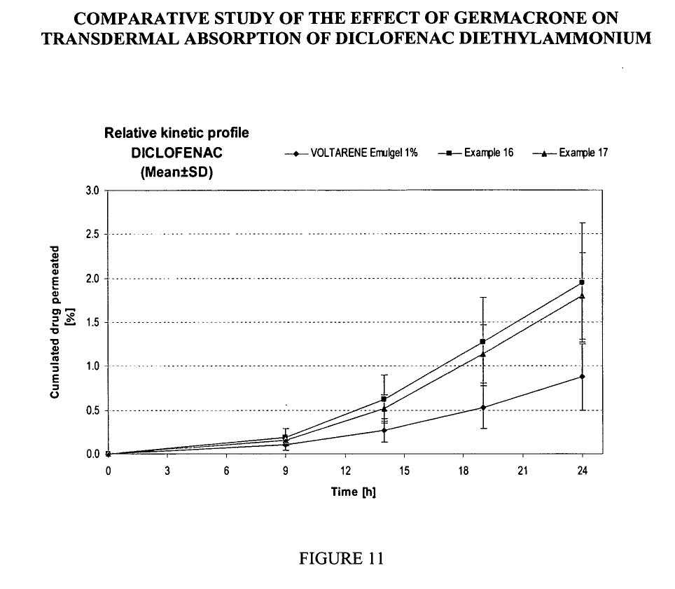

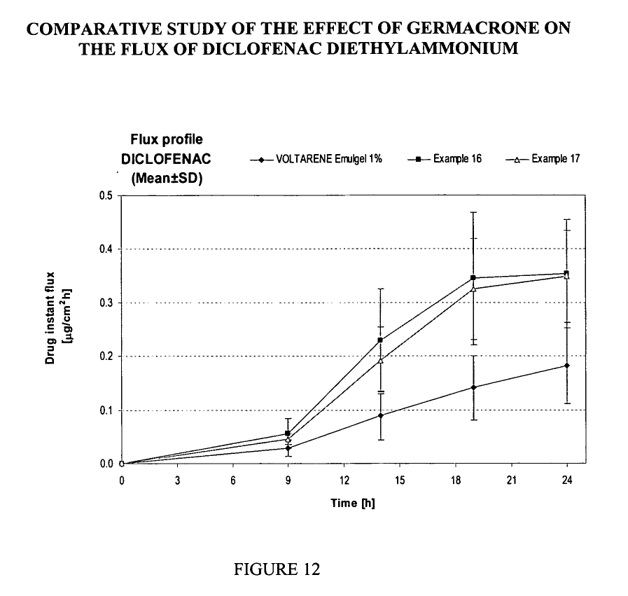

The results are shown in Graphs 11, 12, 13, and 14. As shown in FIG. 11, the transdermal absorption of diclofenac in the gels in accordance with the present invention, Example 16 and Example 17, after 24 hours are at least two times greater as compared to VOLTARENE Emulgel 1%. Additionally, as shown in FIG. 12 the maximum fluxes for the gels in accordance with the present invention are almost two times higher than the flux for VOLTARENE Emulgel 1%.

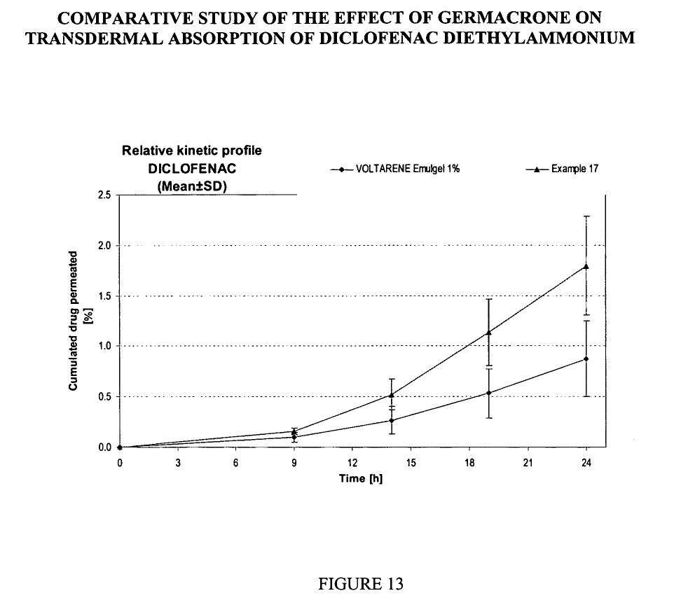

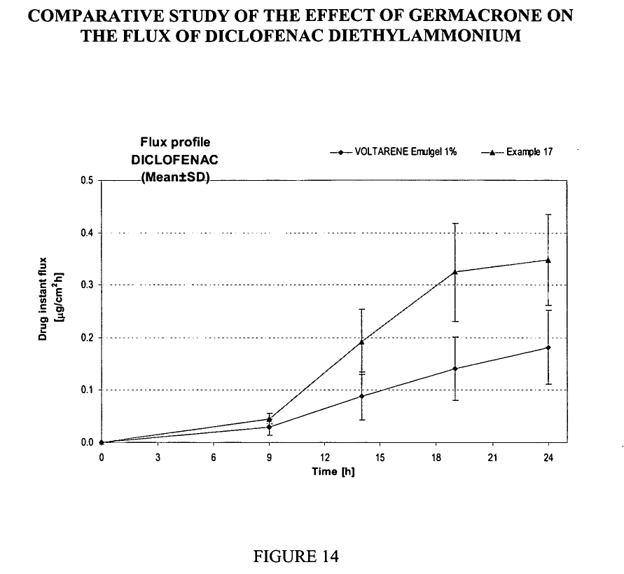

As illustrated in FIGS. 11 and 12, the differences in the relative cumulated diclofenac permeated amounts and in the diclofenac flux profiles for the gel of Example 16 and Example 17, both of which are in accordance with the present invention, are statistically insignificant. Conversely, however, as illustrated in FIGS. 13 and 14, the differences in the relative cumulated diclofenac permeated amounts and in the diclofenac flux profiles for the gel of Example 17 and the VOLTARENE Emulgel 1% are statistically significant. This study illustrates that germacrone enhances skin permeation of diclofenac with similar efficacy as zedoaria oil. As illustrated by the comparison study, the present invention provides a method to double the relative cumulated diclofenac permated amounts and diclofenac flux profiles as compared to presently marketed products. Accordingly, one advantage of the present invention is that it allows one to decrease the dosage of applied diclofenac by 50% which consequently has the effect of decreasing undesirable diclofenac side effects.

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

1. A pharmaceutical formulation comprising:
a therapeutically effective amount of an active agent; and
a permeation enhancer comprising a plant extract of genus Curcuma or a natural or synthetic constituent thereof in an amount sufficient to enhance permeation of the active agent through mammalian dermal or mucosal surfaces.

2. The pharmaceutical formulation of claim 1, wherein the extract is in the form of an oil.

3. The pharmaceutical formulation of claim 2, wherein the oil is an essential oil or a volatile oil.

4. The pharmaceutical formulation of claim 2, wherein the oil is present in an amount between about 0.10% to 15% of the formulation by weight.

5. The pharmaceutical formulation of claim 1, wherein the oil is extracted from the plant by an extraction method including hydrodistillation, solvent extraction, steam distillation, or dense or liquified CO₂ extraction.

6. The pharmaceutical formulation of claim 1, wherein the permeation enhancer is a compound extracted from a species of the genus Curcuma, and further wherein the species is selected from the group including Curcuma zedoaria, Curcuma alismatifolia, Curcuma amada, Curcuma angustifolia, Curcuma aromatica, Curcuma cordata, Curcuma petiolata, Curcuma elata, Curcuma flaviflora, Curcuma gracillima, Curcuma harmandii, Curcuma longa, Curcuma ornata, Curcuma roscoeana, Curcuma sparganifola, and Curcuma thorelii or a combination thereof.

7. The pharmaceutical formulation of claim 1, wherein the natural or synthetic constituents is selected from the group including zedoarofuran, 4-epicurcumenol, neocurcumenol, neocurcumenol, gajutsulactones A and B, zedoarolides A and B, germacrone-4,5-epoxide, germacrone, furanodienone, curzerenone, zedereon, dehydrocurdione, curcumenol, isocurcumenol, curcumenone, curmanolide A, and curmanolide B.

8. The pharmaceutical formulation of claim 1, wherein the plant extract is naturally or synthetically produced.

9. The pharmaceutical formulation of claim 1, wherein the permeation enhancer is present in an amount sufficient to increase at least one of transdermal penetration of the active agent, steady-state flux of the active agent, or transdermal absorption of the active agent.

10. The pharmaceutical formulation of claim 1, wherein the active agent is selected from the group consisting of estrogens, androgens, progestogens, anti-estrogens, anti-androgens, anti-progestogens, sympathomimetics, sympatholytics, parasympathomimetics, parasympatholytics, ganglioplegics, local anesthetics, myorelaxants, antihypertensives, diuretics, cardiotonics, anti-arythmics, anti-angina drugs, cerebral and peripheric vasodilatators, anti-migraine drugs, anti-histaminic drugs, anti-asthma drugs, thrombolytics, general anesthetics, opianalgesics, anxiolytics, antidepressants, neuroleptics, anti-Parkinson drugs, anti-convulsive drugs, hypothalamo-hypophysis regulators, hypo and hyperthyroidics, corticosteroids, glycemia regulators, hypolipidemia drugs, phosphocalcic metabolism regulators, analgesics, antipyretics, antidiabetic agents, antiepileptics, anti-inflammatory drugs, anti-acids, antisecretive gastric drugs, laxatives, gastric mucosa protectors, gastric motricity modulators, bile salts adsorbants, chelators, gall stone dissolvants, anti-anemia drugs, cutaneous diseases drugs, and dermatological drugs, antiparasit drugs, antibiotics, penicillins, cephalosporins, aminosids, polypeptides, sulfamides, diaminopyrimidines, tetracyclins, chloramphenicol, thiamphenicol, macrolides, vancomycin, teicoplanin, rifampicin, fusidic acid, 5-nitro-imidazoles, lincosamides, quinolones, anticancer drugs, anti virus drugs, antiprotozoal drugs, antimalarials, antelmintics and antifungus drugs.

11. The pharmaceutical formulation of claim 1, wherein the weight ratio of permeation enhancer to active agent is between about 10:1 to 1:10.

12. The pharmaceutical formulation of claim 1, wherein the formulation further comprises a delivery vehicle comprising at least one of a monoalkyl ether of diethylene glycol, a polyalcohol, a C₂ to C₄ alcohol, a tetraglycol furol or water.

13. The pharmaceutical formulation of claim 12, wherein the C₂ to C₄ alcohol is ethanol, the polyalcohol is propylene glycol, the monoalkyl ether of diethylene glycol is monoethyl ether of diethylene glycol, and the tetraglycol furol is glycofurol.

14. The pharmaceutical formulation of claim 1, further comprising an agent selected from the group consisting of: gelling agents, pH regulators, neutralizing agents, preservatives, antioxidants, buffers, humectants, sequestering agents, moisturizers, surfactants, emollients, solubilizers, solvents, emulsifiers, skin protectants, essential oils, fragrances, flavors, and any combinations thereof.

15. The pharmaceutical formulation of claim 14, wherein the gelling agent is selected from the group consisting of crosslinked acrylic acid polymers, polyethylene oxides, polyoxyethylene-polyoxypropylene copolymers, sodium polyacrylates, acrylic acid and alkyl methacrylate copolymers, polyvinylalcohols, polyvinylacetates, hydroxypropylcellulose, hydroxyethyl cellulose, hydroxypropyl methylcellulose, hydroxypropyl methylcellulose phthalate, methyl cellulose, polyethylene glycols, polyvidones, polyacrylates, polyacrylamides or a mixture comprising polyacrylamide, aliphatic isoparaffin, and polyoxythelene alkyl ether, dextranes, silicones, carageenans, methylvinyl ether-maleic acid anhydride copolymers, pectines, gum, tragacan gum, xanthan gum, alginates, and gelatin.

16. The pharmaceutical formulation of claim 1, wherein the formulation is in the form of a solution, gel, lotion, cream, spray, aerosol, suppository, jelly, ointment, emulsion, suspension, foam, liposomal system, microsphere, nanosphere, microcapsule, nanocapsule, lacquer, patch, film, bandage, occlusive dressing or a combination thereof.

17. A method for increasing the flux of a drug through mammalian dermal or mucosal surfaces, the method comprising applying to a mammalian subject in need of the drug, the formulation of claim 1.

18. The method of claim 17, wherein the active agent is administered via buccal, nasal, oral, skin, rectal, vaginal, auricular, ophthalmic and mucosal routes.

19. The method of claim 17, wherein the species is selected from the group including Curcuma zedoaria, curcuma alismatifolia, curcuma amada, curcuma angustifolia, curcuma aromatica, curcuma cordata, curcuma petiolata, curcuma elata, curcuma flaviflora, curcuma gracillima, curcuma harmandii, curcuma longa, curcuma ornata, curcuma roscoeana, curcuma sparganifola, and curcuma thorelii or a combination thereof, and further wherein the natural or synthetic constituent is selected from the group including zedoarofuran, 4-epicurcumenol, neocurcumenol, neocurcumenol, gajutsulactones A and B, zedoarolides A and B, germacrone-4,5-epoxide, germacrone, furanodienone, curzerenone, zedereon, dehydrocurdione, curcumenol, isocurcumenol, curcumenone, curmanolide A, and curmanolide B.

20. The method of claim 17, wherein the plant extract is Curcuma zedoaria, and further wherein the Curcuma zedoaria is in the form of an oil.

21. The method of claim 17, wherein the active agent is oxybutynin, progesterone or diclofenac diethylamonium.

22. The method of claim 20, wherein the weight percent ratio of Curcuma zedoaria oil to active agent is between about 10:1 to 1:10.

23. The method of claim 20, wherein the zedoaria oil is present in an amount of about 0.1 to 15%.

24. The method of claim 17, wherein the formulation is in the form of extract is in the form of gel, lotion, cream, spray, aerosol, ointment, emulsion, suspension, foam, liposomal system, microsphere, nanosphere, microcapsule, nanocapsule, lacquer, patch, bandage, occlusive dressing or a combination thereof.

25. A system for the delivery of active agents through mammalian dermal or mucosal surfaces, comprising:
a primary permeation enhancer comprising a plant extract of genus Curcuma or a natural or synthetic constituent thereof, and
a secondary permeation enhancer;
wherein the permeation enhancers are present in a combined amount sufficient to enhance permeation of one or more active agents through mammalian dermal or mucosal surfaces.

26. The delivery system of claim 25 wherein the secondary permeation enhancer comprises a C2 to C4 alcohol, an aliphatic alcohol, a polyalcohol, a monoalkyl ether of diethylene glycol, a tetraglycol furol or a mixture thereof.

27. The delivery system of claim 25 wherein the secondary permeation enhancer comprises a mixture of water, a C2 to C4 alcohol, an aliphatic alcohol, or a polyalcohol, and further comprises either a monoalkyl ether of diethylene glycol or a tetraglycol furol.

28. A pharmaceutical formulation comprising:
a therapeutically effective amount of an active agent; and
a permeation enhancer comprising germacrone or a derivative thereof.

29. The pharmaceutical formulation of claim 28, wherein the germacrone is naturally or synthetically produced.

30. The pharmaceutical formulation of claim 28, wherein the germacrone is extracted from a plant selected from the group consisting of Curcuma genus, rhododendron dauricum, thymus vulgaris, ledum groenlandicum, geranium macrorrhizum, citrullus aromatica and myrica gale.

31. The pharmaceutical formulation of claim 28, wherein the active agent is selected from the group consisting of estrogens, androgens, progestogens, anti-estrogens, anti-androgens, anti-progestogens, sympathomimetics, sympatholytics, parasympathomimetics, parasympatholytics, ganglioplegics, local anesthetics, myorelaxants, antihypertensives, diuretics, cardiotonics, anti-arythmics, anti-angina drugs, cerebral and peripheric vasodilatators, anti-migraine drugs, anti-histaminic drugs, anti-asthma drugs, thrombolytics, general anesthetics, opianalgesics, anxiolytics, antidepressants, neuroleptics, anti-Parkinson drugs, anti-convulsive drugs, hypothalamo-hypophysis regulators, hypo and hyperthyroidics, corticosteroids, glycemia regulators, hypolipidemia drugs, phosphocalcic metabolism regulators, analgesics, antipyretics, antidiabetic agents, antiepileptics, anti-inflammatory drugs, anti-acids, antisecretive gastric drugs, laxatives, gastric mucosa protectors, gastric motricity modulators, bile salts adsorbants, chelators, gall stone dissolvants, anti-anemia drugs, cutaneous diseases drugs, and dermatological drugs, antiparasit drugs, antibiotics, penicillins, cephalosporins, aminosids, polypeptides, sulfamides, diaminopyrimidines, tetracyclins, chloramphenicol, thiamphenicol, macrolides, vancomycin, teicoplanin, rifampicin, fusidic acid, 5-nitro-imidazoles, lincosamides, quinolones, anticancer drugs, anti virus drugs, antiprotozoal drugs, antimalarials, antelmintics and antifungus drugs.

32. The pharmaceutical formulation of claim 28, wherein the germacrone present in an amount between about 0.10% to 15% of the formulation by weight.

33. The pharmaceutical formulation of claim 28, wherein the weight ratio of germacrone to active agent is between about 20:1 to 1:20.

34. The pharmaceutical formulation of claim 28, further comprising a delivery vehicle including at least one of a monoalkl ether of diethylene glycol, a polyalcohol, an alkanol, a tetraglycol furol or water.
